(19) United States
(12) Patent Application Publication
(10) Pub. No.: US 2003/0181860 A1
(43) Pub. Date: Sep. 25, 2003

(54) Shieldable needle assembly with biased safety shield

(75) Inventor: Kirk D. Swenson, North Caldwell, NJ (US)

Correspondence Address: Kirk M. Miles, Esq., WEBB ZIESENHEIM LOGSDON ORKIN & HANSON, P.C., 700 Koppers Building, 436 Seventh Avenue, Pittsburgh, PA, 15219-1818, US

(73) Assignee: Becton, Dickinson and Company

(21) Appl. No.: 10/392,137

(22) Filed: Mar. 19, 2003

Related U.S. Application Data
(60) Provisional application No. 60/365,993, filed on Mar. 20, 2002.

(51) Int. Cl. A61M 5/32

(52) U.S. Cl. 604/192

(57) ABSTRACT

The present invention is directed to a needle safety device including a medical device, such as a needle holder or a syringe. The device includes a needle cannula having a puncture tip extending from an end of the medical device. A shield pivotably connected to the medical device is pivotal with respect to the needle cannula between a shielded position encompassing the needle cannula and a retracted position pivotally spaced from the needle cannula. The shield includes a laterally extending lever, and the medical device includes a laterally extending arm. A spring having stored energy for biasing the needle cannula toward the shielded position is positioned between the lever and the arm. Movement of the lever and the arm toward each other pivots the shield to the retracted position, and releasing the lever and arm allows the stored energy of the spring to pivot the shield into the shielded position.

### CROSS REFERENCE TO RELATED APPLICATIONS

This application claims the benefit of U.S. Provisional Application No. 60/365,993 filed on Mar. 20, 2002.

### BACKGROUND OF THE INVENTION

1. Field of the Invention

The present invention relates to a shield for a needle and more particularly to a safety shield assembly that may be used in conjunction with a syringe assembly, a hypodermic needle, a needle assembly, a needle assembly with a needle holder, a blood collection needle, a blood collection set, an intravenous infusion set or other fluid handling devices or assemblies that contain piercing elements.

2. Description of Related Art

Disposable medical devices having piercing elements for administering a medication or withdrawing a fluid, such as hypodermic needles, blood collecting needles, fluid handling needles and assemblies thereof, require safe and convenient handling. The piercing elements include, for example, pointed needle cannulae or blunt ended cannulae.

Safe and convenient handling of disposable medical devices is recognized by those in the medical arts so as to minimize exposure to blood borne pathogens. Safe and convenient handling of disposable medical devices results in the disposal of the medical devices intact.

As a result of this recognition, numerous devices have been developed for shielding needles after use. Many of these devices are somewhat complex and costly. In addition, many of these devices are cumbersome to use in performing procedures. Furthermore, some of the devices are so specific that they preclude use of the device in certain procedures or with certain devices and/or assemblies.

For example, a number of devices incorporate a pivoting shield assembly in which the shield can be pivoted away from the needle during use and pivoted about the needle after use, for protection from the used needle. U.S. Pat. No. 5,603,699 discloses a needle guard assembly which includes a top shield member and a bottom lever member which pivot away from the needle of a syringe in opposing directions. The assembly further includes a series of gears between the top shield member and the bottom lever member, as well as a torsional coil spring mounted therebetween. Such an arrangement is not practically useful due to the complex arrangement of gears and opposing pivoting members. U.S. Pat. No. 5,401,251 discloses a syringe injection system including a hollow needle and a safety cap cover for covering the needle after use. The safety cap cover is attached to the body of the syringe through an elongated extension arm, which pivots the safety cap cover over the needle after use. The extension arm may be attached to the body of the syringe through a spring, which urges the arm and cover into the shielded position. Such an arrangement, however, involves extensive movement of the arm to fully expose the needle, and exposing the needle tip from the safety shield prior to use requires that the user hold the syringe in one hand and grasp the cover near the needle with the other hand, thereby potentially exposing the user to the needle tip.

In view of the foregoing, a need exists for a blood collection set including a shieldable needle assembly that achieves secure and effective shielding of a used needle cannula which is simple and inexpensive to manufacture and easy to operate.

### SUMMARY OF THE INVENTION

The present invention is directed to a shieldable needle assembly, and in particular, to a needle safety device. The needle safety device includes a medical device, such as a blood collection needle holder or a syringe, including a forward or distal end having an opening therethrough and a laterally extending arm. A needle cannula extends from the forward or distal end of the medical device, with the needle cannula including a distal end having a puncture tip. A shield is pivotably connected to the forward or distal end of the medical device. The shield is pivotal with respect to the needle cannula between a shielded position encompassing the distal end of the needle cannula and a retracted or non-shielded position pivotally spaced from the distal end of the needle cannula. The shield includes a laterally extending lever which is adjacent the forward end of the medical device which includes the laterally extending arm. The laterally extending lever and the laterally extending arm are in close proximity and extend away from the same side of the assembly, represented by a plane defined by the longitudinal axis of the needle cannula and a pivot of the shield.

A biasing element extends between the medical device and the shield, for biasing the shield toward the shielded position. The biasing element is capable of storing energy when the shield is in the non-shielded position for biasing the shield to the shielded position. The biasing element may be, for example, a wound coil torsion spring or a leaf spring, including first and second legs at opposite ends thereof. The first leg is in engagement with the laterally extending arm of the medical device and the second leg is in engagement with the laterally extending lever of the shield. The first and second legs may form the laterally extending arm and the laterally extending shield, respectively.

The medical device and the shield may be integrally formed, desirably with the biasing element integral therebetween forming a leaf spring. The laterally extending lever of the shield and the laterally extending arm of the medical device may include interengaging structure for releasably holding the shield in the non-shielded position. Also, the shield may include a needle cannula lock which is movable between a first position and a second position. In the first position, the needle cannula lock is bent, and provides for movement of the shield from the shielded position, such as when the needle device is in a packaged or pre-use state with the shield covering the needle cannula in a reversible shielded position. In the second position, the needle cannula lock prevents movement of the shield from the shielded position, such as when the shield has been pivotably rotated to the non-shielded position and returned to the shielded position. Such a needle cannula lock may include a finger with a needle engaging barb on one side thereof, with the needle engaging barb engaging the needle cannula for preventing pivotal movement of the shield from the shielded position when the needle cannula lock is in the second position, and with the needle cannula releasably engaging an opposite side of the finger thereby allowing for movement of the shield from the shielded position when the needle cannula lock is in the first position.

In one particular embodiment, the needle cannula lock in the first position exerts a biasing force against the needle cannula, biasing the shield toward the non-shielded position, and the biasing element exerts a biasing force greater than the biasing force exerted by the cannula lock for maintaining the shield biased toward the shielded position.

In a further embodiment, the invention is directed to a safety blood collection device including a needle holder as the medical device and having a laterally extending arm, with a needle cannula extending through an opening at the end of the needle holder. The needle cannula includes a non-patient end including a non-patient puncture tip extending within the needle holder, as well as an intravenous end including an intravenous puncture tip extending from the end of the needle holder. A sleeve may extend about the non-patient puncture tip. A shield is pivotably connected to the distal end of the needle holder, and is pivotal with respect to the needle cannula between a shielded position encompassing the distal end of the needle cannula and a non-shielded position pivotally spaced from the distal end of the needle cannula in a similar manner as set forth above, including a laterally extending lever and a biasing element for pivotal movement of the shield.

In a further embodiment, the invention is directed to a safety needle assembly for use in conjunction with a medical device such as a needle holder or a syringe. Such a safety needle assembly includes a base hub having an internal opening therethrough, and a laterally extending arm extending from the outer wall of the base hub. A needle cannula extends from a forward or distal end of the base hub, with the needle including a puncture tip and an internal lumen in communication with the internal opening of the base hub. A shield is pivotably connected to the base hub, and is pivotal with respect to the needle cannula in a similar manner as described above, including a laterally extending lever and a biasing element for pivotal movement of the shield. In such an embodiment, the base hub preferably includes structure for mating with a medical device, such as a blood collection needle holder or a syringe.

In a particular embodiment, the needle cannula of such a safety needle assembly includes a non-patient end having a non-patient puncture tip extending from one end of the base hub, and an intravenous end including an intravenous puncture tip extending from the other end of the base hub. A sleeve may extend about the non-patient puncture tip. Such a safety needle assembly is particularly adapted for mating with a blood collection needle holder through the base hub, such as through threaded engagement.

In a further embodiment, the invention relates to a safety needle assembly which includes a base hub including a proximal end and a distal end with a needle having a puncture tip extending from the distal end of the hub, a laterally extending arm adjacent the distal end of the hub and extending laterally from the hub, and a laterally extending lever extending laterally from the distal end of the hub. A hinge section is formed at the intersection of the laterally extending arm and the laterally extending lever defining an acute angle therebetween. The hinge section further defines a pivot axis for pivotal movement of the laterally extending lever with respect to the laterally extending arm between a first position and a second position with the second position defining an angle which is more acute than the first position. The assembly further includes a shield which is integral with the laterally extending lever and which extends toward the distal end of the needle for encompassing the distal end of the needle when the hinge is in the first position. As such, pivotal movement of the laterally extending lever from the first position to the second position causes biasing energy to accumulate between the laterally extending arm and the laterally extending lever, such that the biasing energy biases the laterally extending lever to the first position for encompassing the distal end of the needle.

Desirably, the biasing energy accumulates in a spring element, causing a bending moment between the laterally extending arm and the laterally extending lever. More desirably, the biasing energy accumulates in the hinge section, with the hinge section forming a leaf spring.

### DESCRIPTION OF THE DRAWINGS

FIG. 1 is a perspective view of a needle assembly attached to a needle holder in accordance with one embodiment of the present invention, with the shield in a first packaging position prior to use;

FIG. 2 is a cross section of the needle assembly of FIG. 1;

FIG. 3 is a cross section of the needle assembly of FIG. 1 shown in a second position with the shield pivoted away from the needle for use;

FIG. 4 is a transverse cross section taken along lines 4-4 of FIG. 2, showing the cannula lock in a first packaging position prior to use;

FIG. 5 is a transverse cross section of the cannula lock in locked position when the needle assembly is in a shielded position;

FIG. 6 is a perspective view of the needle assembly of FIG. 1 shown in use in a blood collection procedure;

FIG. 7 is a perspective view of a needle assembly attached to a needle holder in accordance with a further embodiment of the present invention;

FIG. 8 is a perspective view of a needle assembly as in FIG. 1 shown with a needle cover covering the needle in an alternate packaging embodiment prior to use;

FIG. 9 is a perspective view of a double ended needle assembly for attachment to a needle holder in accordance with a further embodiment of the invention;

FIG. 10 is a cross section of the needle assembly of FIG. 9 shown in a sampling position;

FIG. 11 is a cross section of the needle assembly of FIG. 9 shown in a shielded position after use; and

FIG. 12 is a cross section of a needle assembly for attachment to a syringe in accordance with a further embodiment of the present invention.

### DETAILED DESCRIPTION

While this invention is satisfied by embodiments in many different forms, there is shown in the drawings and will herein be described in detail, the preferred embodiments of the invention, with the understanding that the present disclosure is to be considered as exemplary of the principles of the invention and is not intended to limit the invention to the embodiments illustrated. Various other modifications will be apparent to and readily made by those skilled in the art without departing from the scope and spirit of the invention. The scope of the invention will be measured by the appended claims and their equivalents.

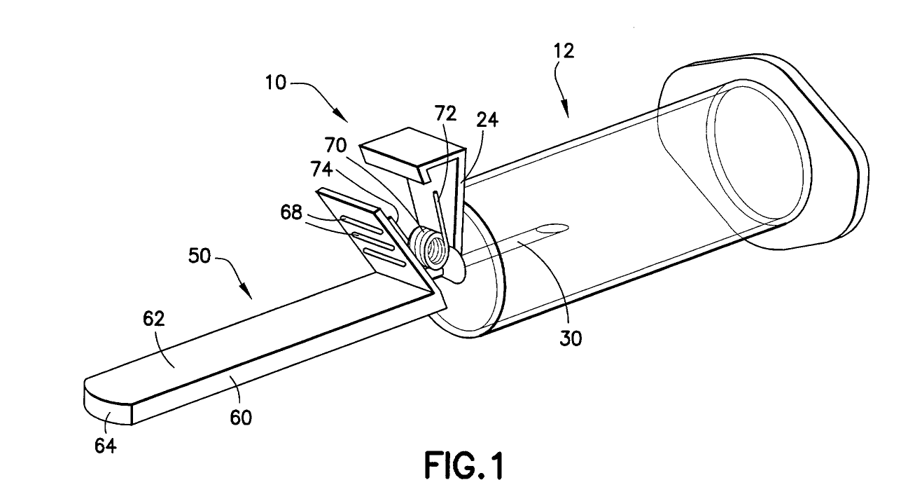

Referring to the drawings in which like reference characters refer to like parts throughout the several views thereof, FIGS. 1-3 illustrate a needle safety device in accordance with the present invention and the related features, in the form of a blood collection device 10. The present invention is generally described in terms of a needle safety device in the form of such a blood collection device, and encompasses such a device as well as a shieldable safety needle assembly for use in such a device.

The safety needle device includes a medical device, such as a needle holder 12 for use in blood collection procedures, as shown in FIGS. 1-3. While described herein in terms of a blood collection device including a needle holder, the needle safety device of the present invention may incorporate other medical devices used in connection with a needle, such as a syringe assembly, a hypodermic needle, a needle assembly, a blood collection set, an intravenous infusion set or other fluid handing devices or medical device assemblies that contain piercing elements.

Needle holder 12 includes a generally tubular body 14 having proximal end 16 and distal end 18 at opposing ends thereof, with internal opening 20 extending therebetween. Proximal end 16 includes a flange 22, which may extend circumferentially about proximal end 16. Distal end 18 includes a distal opening 19 extending through tubular body 14 into internal opening 20.

Needle holder 12 further includes a laterally extending arm 24 which extends laterally away from tubular body 14 adjacent distal end 18. Laterally extending arm 24 extends laterally from axis X defining blood collection device 10, and may be a generally planar structure. Laterally extending arm 24 is desirably integrally formed with needle holder 12.

Blood collection device 10 further includes needle cannula 30 extending from distal end 18 of needle holder 12. The needle cannula 30 has a proximal end 32 and an opposing distal end 34. The needle cannula 30 defines an internal lumen 36 extending through the needle cannula 30 from proximal end 32 to distal end 34. Distal end 34 of needle cannula 30 is beveled to define a sharp puncture tip at intravenous puncture tip 38. Intravenous puncture tip 38 is provided for insertion into a patient's blood vessel, such as a vein, and is, therefore, designed to provide ease of insertion and minimal discomfort during venipuncture. As FIGS. 1-3 depict blood collection device 10 with needle holder 12 as a medical device for blood collection, the proximal end 32 of needle cannula 30 further includes non-patient puncture tip 40. Non-patient puncture tip 40 is provided for puncturing of an evacuated tube, for example, during a blood collection procedure, and therefore includes a sharp puncture tip. Internal lumen 36 extends between intravenous puncture tip 38 and non-patient puncture tip 40. An elastomeric sleeve 42 covers the non-patient puncture tip 40 at the proximal end 32.

As noted above, while such a description relates to a medical device in the form of a needle holder for blood collection, other medical devices for use with a needle may be provided through the present invention, including a syringe.

Blood collection device 10 further includes a shield 50 pivotably connected to needle holder 12 at distal end 18. Shield 50 comprises a rearward end 52 and a forward end 54. Forward end 54 of shield 50 includes a slot or longitudinal opening 56 formed by sidewalls 58 and 60 that extend downwardly from top section 62 and run substantially opposite of one another in parallel along the length of slot 56 towards forward endwall 64. Shield 50 further includes a laterally extending lever 66 which extends laterally away from top section 62 of shield 50 at rearward end 52, adjacent distal end 18 of needle holder 12. Laterally extending lever 66 extends laterally away from axis X defining the blood collection device 10, and may be a generally planar structure integrally formed with shield 50. Bumps or ribs 68 may be provided on a surface of laterally extending lever 66 for providing a tactile surface for engagement with a user's finger.

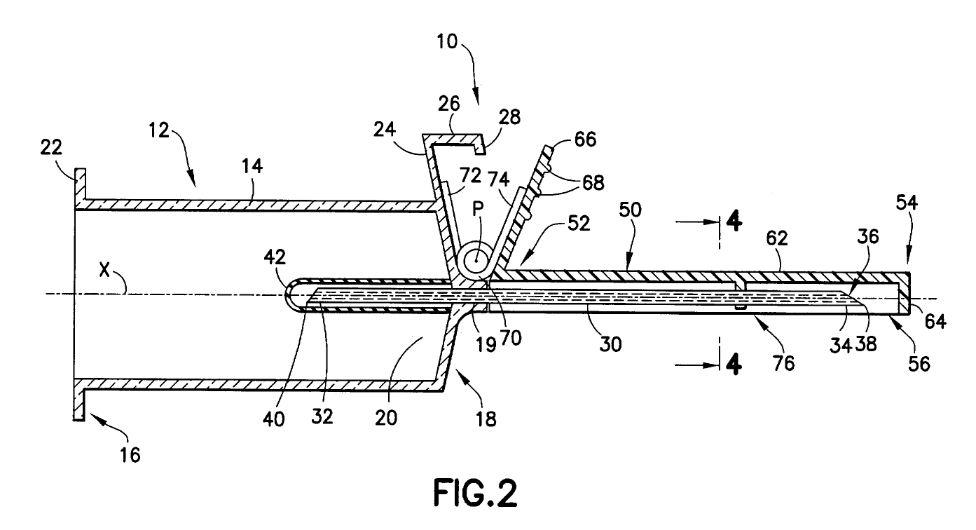

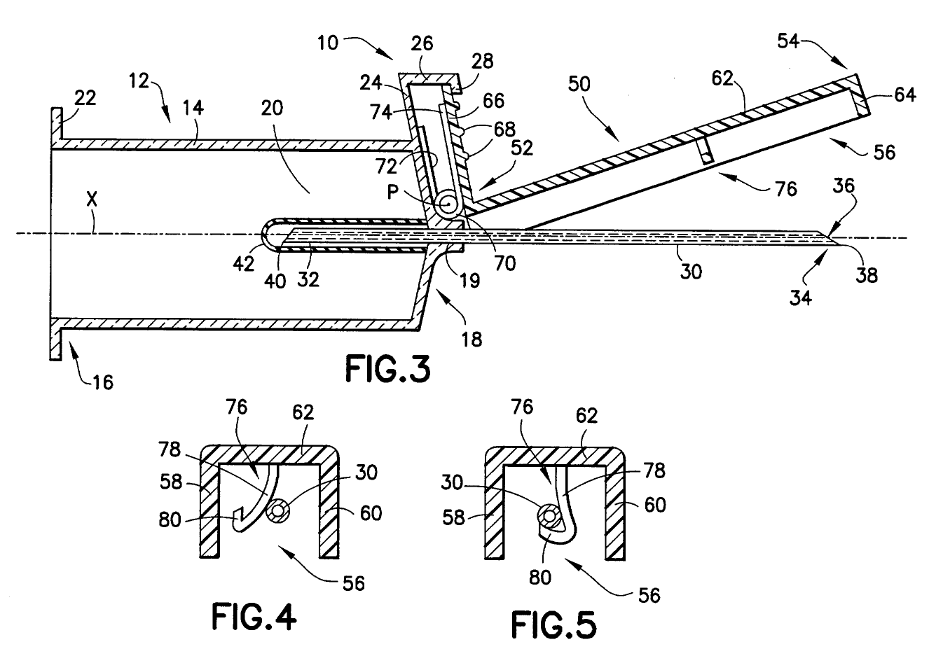

Shield 50 is pivotal with respect to needle cannula 30 about a pivoting point P between a retracted or non-shielded position as shown in FIG. 3 in which shield 50 is pivotally spaced from distal end 34 of needle cannula 30, and a shielded position as shown in FIG. 2 in which the distal end 34 of needle cannula 30 is encompassed within slot 56 of shield 50.

Blood collection device 10 further includes a biasing element, such as spring 70, extending between laterally extending arm 24 of needle holder 12 and laterally extending lever 66 of shield 50. Spring 70 provides a biasing force between needle holder 12 and shield 50, and includes stored energy for biasing shield 50 toward the shielded position encompassing needle cannula 30. Spring 70 may be a wound torsion spring such as a coil spring shown in FIGS. 1-3, a compression spring, or a leaf spring.

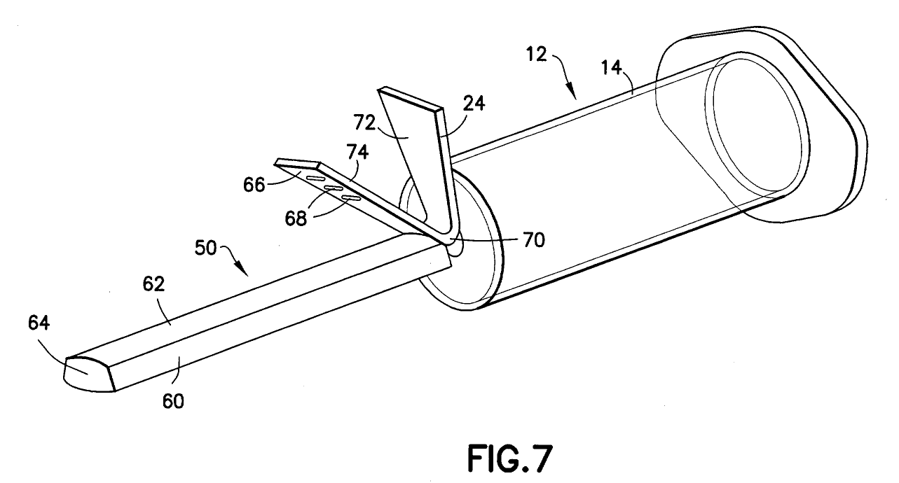

Spring 70 includes a first leg 72 and a second leg 74 at opposing ends. First leg 72 is in engagement with laterally extending arm 24 of needle holder 12, and second leg 74 is in engagement with laterally extending lever 66 of shield 50. As shown in FIGS. 1-3, such engagement may be achieved with first leg 72 and second leg 74 lying against and/or fixedly adhered to the planar surfaces of laterally extending arm 24 and laterally extending lever 66, respectively. Alternatively, first leg 72 and second leg 74 of spring 70 may comprise laterally extending arm 24 and laterally extending lever 66, such as depicted in FIG. 7, with first leg 72 and second leg 74 of the leaf spring forming laterally extending arm 24 and laterally extending lever 66, respectively. As such, laterally extending arm 24 and laterally extending lever 66 intersect to form a hinge section at spring 70, with shield 50 integral with the laterally extending lever 66 and extending toward the distal end of needle cannula 30. The leaf spring acts as a biased hinge between laterally extending arm 24 and laterally extending lever 66. In such an embodiment, needle holder 12, shield 50 and spring 70 are preferably unitarily formed as an integral part.

Laterally extending arm 24 and laterally extending lever 66 extend away from the same side of a plane defined by the longitudinal axis X of blood collection device 10 and the pivoting point P of shield 50. Moreover, first leg 72 and second leg 74 of spring 70 also extend away from the same side of a plane defined by the longitudinal axis X of blood collection device 10 and the pivoting point P of shield 50. Such an arrangement provides first leg 72 and second leg 74, and therefore laterally extending arm 24 and laterally extending lever 66 in engagement therewith, in close approximation with one another for ease of movement therebetween, as will be described in more detail herein.

Desirably, laterally extending arm 24 of needle holder 12 and laterally extending lever 66 of shield 50 include interengaging structure for releasably holding shield 50 in the retracted position. Such interengaging structure may be provided through a latch mechanism, such as by providing laterally extending arm 24 with a planar surface 26 and a latch 28, for releasably engaging the top edge of laterally extending lever 66 of shield 50, as shown in FIG. 3. It is contemplated that other releasable engaging arrangements may be used, for example, by providing laterally extending lever 66 with such a latching mechanism for engagement with laterally extending arm 24.

Shield 50 may include means for trapping the needle cannula 30 in slot 56, such as a needle cannula lock 76. As shown in FIGS. 4 and 5, such needle cannula lock 76 includes a finger 78 that extends from an interior portion of top section 62, with a needle engaging barb 80 extending from one side thereof. Finger 78 of needle cannula lock 76 is a resiliently flexible material. The needle cannula lock 76 is movable between a first position shown in FIG. 4, permitting pivotal movement of shield 50, and a second position shown in FIG. 5, preventing pivotal movement of shield 50. More particularly, when shield 50 is in a first position, such as during packaging prior to use, finger 78 of needle cannula lock 76 is in a first bent position, with needle cannula 30 sitting against one side of finger 78. The resilient flexible nature of finger 78 exerts a biasing force against needle cannula 30 with finger 78 in this first position, biasing shield 50 toward the retracted position. Spring 50, however, exerts a biasing force in the opposing direction biasing the shield toward the shielded position, which biasing force of spring 50 is greater than the biasing force of finger 78 in this first position, thereby maintaining the shield biased toward the shielded position, for packaging. The resilient nature of finger 78 causes finger 78 to move from the first bent position to a second relaxed or rest position when needle cannula 30 is out of engagement therewith, such as when shield 50 is pivoted to the retracted position. When shield 50 is again pivoted to the shielded position, such as when a procedure is completed, finger 78 slightly deflects to the opposing side of finger 78, whereby the needle is permanently trapped by needle engaging barb 80. Such an arrangement provides a needle cannula lock which is automatically movable between a first position, which permits movement of shield 50 to a retracted position, and a second position, which prevents movement of shield 50 from the shielded position. It is further contemplated that the needle cannula lock may include a mechanism for mechanically engaging the lock when shield 50 is in a retracted position.

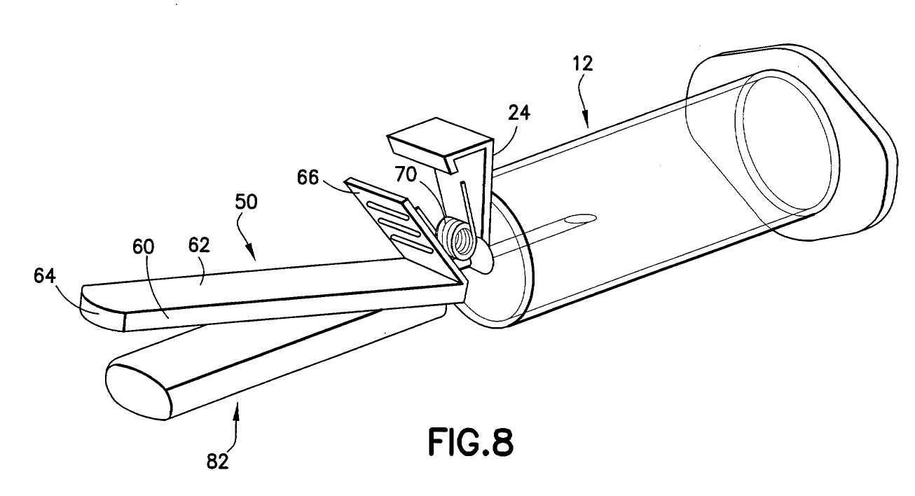

As noted, such a needle cannula lock may provide blood collection device 12 in a one time reversible shielded position during packaging. A removable protective cover may further be provided along slot 56 in this packaged condition. Alternatively, as depicted in FIG. 8, shield 50 may be slightly retracted during packaging, with a removable protective needle cover such as rigid sleeve 82 positioned over distal end 34 of needle cannula 30 for protection from intravenous puncture tip 38 during packaging and prior to use.

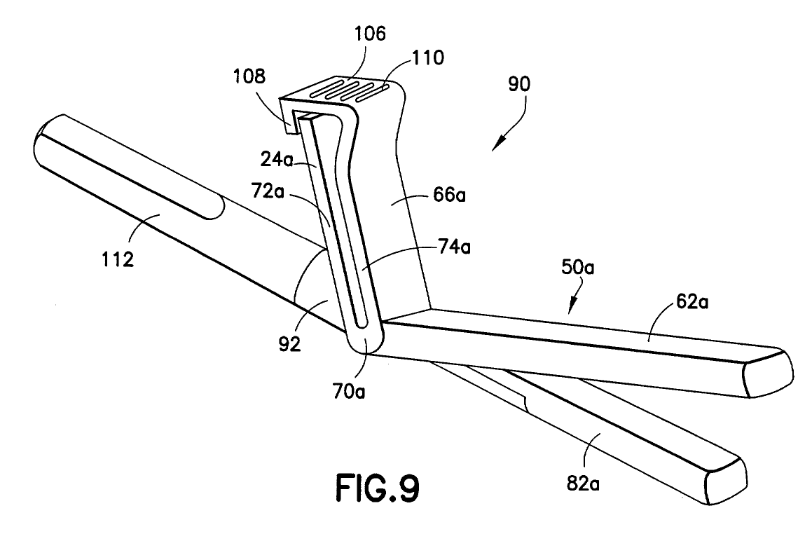

FIGS. 9-12 depict a further embodiment of the invention that includes many components which are substantially identical to the components of FIGS. 1-8. Accordingly, similar components performing similar functions will be numbered identically to those components of FIGS. 1-8, except that a suffix “a” will be used to identify those similar components in FIGS. 9-12.

The embodiment of FIGS. 9-12 includes safety needle assembly 90 for use with conventional medical devices, such as conventional needle holders for blood collection, syringes, and the like. As shown in FIGS. 9-12, the safety needle assembly 90 includes a needle cannula 30a, a shield 50a and a biasing element in the form of spring 70a, as set forth in the embodiment described above. In the embodiment of FIGS. 9-12, the safety needle assembly 90 is an independent component for attachment to a medical device, and further includes a base hub 92 for providing such attachment.

Base hub 92 includes a proximal end 94 and distal end 96, with an internal opening 98 extending therethrough. Needle cannula 30a extends through internal opening 98 of base hub 92, with proximal end 32a of needle cannula 30a extending from proximal end 94 of base hub 92, and distal end 34a of needle cannula 30a extending from distal end 96 of base hub 92. Base hub 92 further includes laterally extending arm 24a which extends laterally away from base hub 92 adjacent distal end 96, in a similar manner as with laterally extending arm 24 described above in connection with the embodiment of FIGS. 1-8.

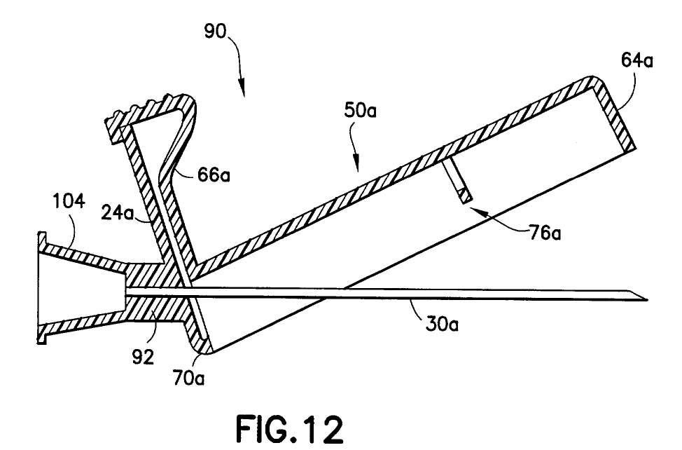

Base hub 92 may include a threaded end 100 at the proximal end thereof. Preferably, threaded end 100 comprises male threads 102 for mounting the hub on a conventional needle holder. Alternatively, as shown in FIG. 12, base hub 92 may include a female luer fitting 104 at the proximal end thereof for attachment with a male luer fitting, and may include additional luer lugs for attachment with a luer collar, such as a syringe luer collar.

Base hub 92 is interconnected with shield 50a in a similar manner as the interconnection between needle holder 12 and shield 50 in the previously described embodiment of FIGS. 1-8. Desirably, base hub 92 and shield 50a are integrally formed. Spring 70a is provided between base hub 92 and shield 50a, and is desirably a leaf spring, with first leg 72a and second leg 74a forming laterally extending arm 24a of base hub 92 and laterally extending lever 66a of shield 50a. In such an embodiment, interengaging structure between laterally extending arm 24a of base hub 92 and laterally extending lever 66a of shield 50a is desirably provided through lever planar surface 106 and latch 108 of laterally extending lever 66a, which engage a top edge of laterally extending arm 24a. Lever planar surface 106 preferably includes ribs 110 as a tactile surface for a user.

Safety needle assembly 90 may be packaged with a removable protective cover provided along slot 56a as discussed above, or, as depicted in FIG. 9, shield 50a may be slightly retracted during packaging, with a removable protective needle cover such as rigid sleeve 82a positioned over distal end 34a of needle cannula 30a for protection from intravenous puncture tip 38a during packaging and prior to use. In addition, a second rigid sleeve 112 may be provided over proximal end 32a of needle cannula 30a for protection from non-patient puncture tip 40a.

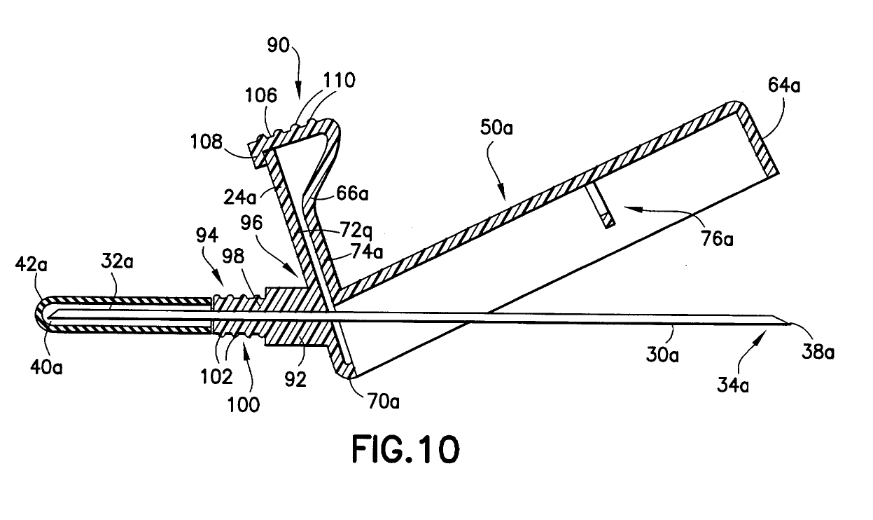

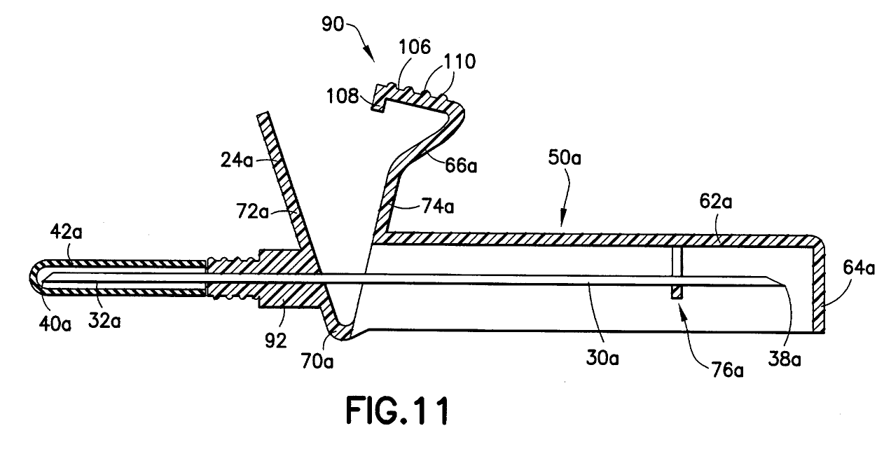

With the basic structure of the assembly now described, operation of the needle safety device will be described with reference to the blood collection device 10 shown in FIGS. 1-7. In use, the blood collection device 10 is provided as depicted in FIG. 1, with shield 50 in a shielded position and with finger 78 of the needle cannula lock 76 in a first bent position as in FIG. 4. A protective covering may be provided over slot 56, which protective covering is removed in preparation for use. To prepare for use of the blood collection device 10, the user applies a force between laterally extending arm 24 and laterally extending lever 66, such as by pinching them together between the user's fingers, thereby causing shield 50 to pivot about the pivoting point P to a retracted or non-shielded position. This pinching causes the hinge section between laterally extending arm 24 and laterally extending lever 66 to move from a first position defining an acute angle as shown in FIGS. 2 and 11, to a second position defining an acute angle which is more acute that the angle defined by this hinged section in the first position, as shown in FIGS. 3 and 10. Such pivoting causes biasing energy to accumulate between laterally extending arm 24 and laterally extending lever 66 and be stored in the biasing element of spring 70, thereby causing a bending moment between laterally extending arm 24 and laterally extending lever 66. Such pivoting also causes finger 78 of needle cannula lock 76 to move from the first bent position to a second relaxed position. The top edge of laterally extending lever 66 is engaged by latch 28 of laterally extending arm 24, thereby locking shield 50 in the retracted position with intravenous puncture tip 38 exposed for use. In embodiments including a rigid sleeve 82 as a packaging cover over the distal end of the needle cannula as depicted in FIG. 8, the user grasps the assembly in one hand and applies a pinching force in a similar manner as described above. With the other hand, the user removes the rigid sleeve 82 from needle cannula 30, thereby exposing intravenous puncture tip 38 for use.

The medical practitioner then sterilizes the intended area of puncture on the patient's body, and can then urge intravenous puncture tip 38 at distal end 34 of needle cannula 30 into a targeted blood vessel of a patient. An appropriate medical procedure can then be conducted. Upon completion of the procedure, such as when all desired samples have been drawn, needle cannula 30 is withdrawn from the patient, and shielding of the needle can be accomplished. In particular, the user lifts latch 28 to release the top edge of laterally extending lever 66, thereby releasing the interengagement with shield 50. The stored energy of spring 70 causes shield 50 to pivot about pivoting point P to the shielded position. Hence, shield 50 safely shields and encompasses needle cannula 30 and intravenous puncture tip 38. In addition, needle engaging barb 80 of needle cannula lock 76 engages needle cannula 30, as shown in FIG. 5, thereby preventing any further pivotal movement of shield 50 to the retracted position. The needle safety device can then be safely discarded.

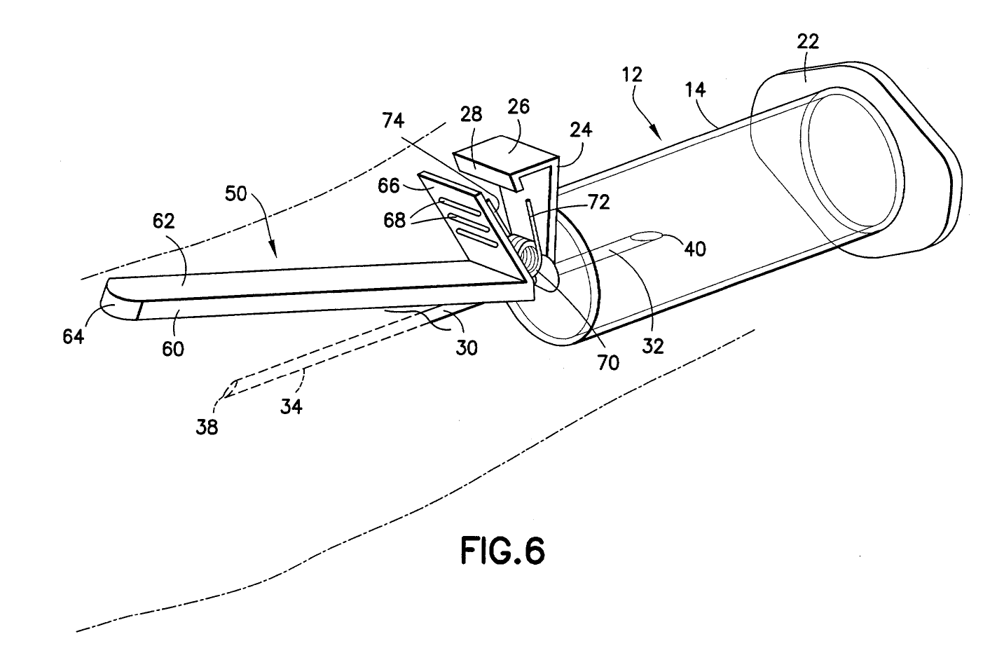

Shielding of the needle may also be passively accomplished through the present invention. In particular, it is noted that activation of the safety shield may be accomplished while venipucture is maintained, that is while intravenous puncture tip 38 of needle cannula 30 is maintained within the blood vessel of the patient. For example, once intravenous puncture tip 38 of the needle cannula 30 is inserted into a blood vessel in the patient's body (i.e., venipuncture), the user can lift latch 28 to release the top edge of laterally extending lever 66, thereby releasing the interengagement with shield 50, and causing shield 50 to pivot around pivoting point P due to the biasing force of spring 70. Since intravenous puncture tip 38 is within the patient's blood vessel, such pivotal movement of shield 50 will terminate when the forward end 54 of shield 50 contacts the skin of the patient, as shown in FIG. 6. Upon removal of intravenous puncture tip 38 from the patient's blood vessel, shield 50 will continue in its pivotal rotation to the shielded state, thereby shielding intravenous puncture tip 50 and needle cannula 30 and locking needle cannula lock 76 in place.

In the alternate embodiment of FIGS. 9-12, the safety needle assembly 90 is assembled with an appropriate medical device, such as a needle holder, prior to use. For example, second rigid sleeve 112 is removed, and the needle holder is screwed onto base hub 92 through threads 102. The user then removes rigid sleeve 82a from distal end 34a of needle cannula 30a in a similar manner as described above, thereby exposing intravenous puncture tip 38a for use, and pivoting and locking shield 50a in the retracted position. The safety needle assembly can then be used for an appropriate medical procedure and the shield 50a can thereafter be pivoted to the shielded position, as discussed above.

The shield and hub of the safety shield assembly of the present invention are comprised of moldable parts which can be mass produced from a variety of materials including, for example, polyethylene, polyvinyl chloride, polystyrene or polyethylene and the like. Materials will be selected which will provide the proper covering and support for the structure of the invention in its use, but which will provide also a degree of resiliency for the purpose of providing the cooperative movement relative to the shield and the hub of the assembly.

While the needle assembly of the present invention has been described in terms of one embodiment for use in connection with a blood collection system, it is further contemplated that the needle assembly could be used with other medical procedures, such as in conjunction with conventional intravenous infusion sets, which are well known in the art for use with needle assemblies.

What is claimed is:

1. A needle safety device comprising:
a medical device including a distal end having an opening therethrough and a laterally extending arm;
a needle cannula extending from said distal end of said medical device, said needle cannula including a distal end having a puncture tip;
a shield pivotably connected to said medical device at said distal end thereof, said shield being pivotal with respect to said needle cannula between a shielded position encompassing said distal end of said needle cannula and a non-shielded position pivotally spaced from said distal end of said needle cannula, said shield including a laterally extending lever adjacent said laterally extending arm of said medical device, said laterally extending lever and said laterally extending arm extending away from the same side of a plane defined by the longitudinal axis of said needle cannula and a pivot of said shield; and
a biasing element extending between said laterally extending arm of said medical device and said laterally extending lever of said shield for biasing said shield toward the shielded position, said biasing element capable of storing energy when said shield is in said non-shielded position for biasing said shield to said shielded position.

2. A needle safety device as in claim 1, wherein said laterally extending lever of said shield and said laterally extending arm of said medical device include interengaging structure for releasably holding said shield in said non-shielded position.

3. A needle safety device as in claim 1, wherein said shield includes a needle cannula lock which in a first position will provide for movement of said shield from said shielded position and in a second position will prevent movement of said shield from said shielded position.

4. A needle safety device as in claim 3, wherein said needle cannula lock is automatically movable from said first position to said second position.

5. A needle safety device as in claim 3, wherein said needle cannula lock includes a finger with a needle engaging barb on one side thereof.

6. A safety needle device as in claim 5, wherein with said needle cannula lock in said second position said needle engaging barb engages said needle cannula preventing pivotal movement of said shield from said shielded position and in said first position said needle cannula releasably engages an opposite side of said finger allowing for movement of said shield from said shielded position.

7. A needle safety device as in claim 1, wherein said medical device and said shield are integral.

8. A needle safety device as in claim 1, wherein said biasing element comprises a spring extending between said medical device and said shield for biasing said shield toward the shielded position, said spring including first and second legs at opposite ends thereof, said first and second legs extending away from the same side of a plane defined by the longitudinal axis of said needle cannula and a pivot of said shield, said first leg in engagement with said laterally extending arm of said medical device and said second leg in engagement with said laterally extending lever of said shield.

9. A needle safety device as in claim 8, wherein said spring is a wound torsion spring.

10. A needle safety device as in claim 1, wherein said medical device and said shield are integral, and wherein said biasing element comprises a leaf spring with a first leg forming said laterally extending arm of said medical device and a second leg forming said laterally extending lever of said shield.

11. A needle safety device as in claim 1, wherein said medical device comprises a syringe.

12. A needle safety device as in claim 1, wherein said medical device comprises a blood collection needle holder.

13. A needle safety device as in claim 12, wherein said needle cannula includes a proximal end extending within said needle holder and including a non-patient puncture tip, and a distal end extending from said distal end of said needle holder and including an intravenous puncture tip.

14. A needle safety device as in claim 13, further comprising a sleeve extending about said non-patient puncture tip.

15. A needle safety device as in claim 1, wherein said laterally extending lever of said shield includes a tactile surface for engagement with a user's finger.

16. A needle safety device as in claim 1, further comprising a packaging needle cover covering said distal end of said needle.

17. A safety blood collection device comprising:
a needle holder including a distal end having an opening therethrough and a laterally extending arm;
a needle cannula extending through said opening of said needle holder, a proximal end of said needle cannula extending within said needle holder and including a non-patient puncture tip, a distal end of said needle cannula extending from said distal end of said needle holder and including an intravenous puncture tip;
a shield pivotably connected to said needle holder at said distal end thereof, said shield being pivotal with respect to said needle cannula between a shielded position encompassing said distal end of said needle cannula and a non-shielded position pivotally spaced from said distal end of said needle cannula, said shield including a laterally extending lever adjacent said laterally extending arm of said medical device, said laterally extending lever and said laterally extending arm extending away from the same side of a plane defined by the longitudinal axis of said needle cannula and a pivot of said shield; and
a biasing element extending between said laterally extending arm of said needle holder and said laterally extending lever of said shield for biasing said shield toward the shielded position, said biasing element adapted for storing energy when said shield is in said non-shielded position for biasing said shield to said shielded position.

18. A safety blood collection device as in claim 17, wherein said laterally extending lever of said shield and said laterally extending arm of said needle holder include interengaging structure for releasably holding said shield in said retracted position.

19. A needle safety device comprising:
a medical device including a distal end having an opening therethrough and a laterally extending arm;
a needle cannula extending from said distal end of said medical device, said needle cannula including a distal end having a puncture tip;
a shield pivotably connected to said medical device at said distal end thereof, said shield being pivotal with respect to said needle cannula between a shielded position encompassing said distal end of said needle cannula and a non-shielded position pivotally spaced from said distal end of said needle cannula, said shield including a laterally extending lever adjacent said laterally extending arm of said medical device, said laterally extending arm of said medical device and said laterally extending lever of said shield including interengaging structure for maintaining said shield in said non-shielding position, said shield further including a needle cannula lock which in a first position will provide for movement of said shield from said shielded position and in a second position will prevent movement of said shield from said shielded position; and
a biasing element extending between said laterally extending arm and said laterally extending lever for biasing said shield toward the shielded position, said biasing element adapted for storing energy when said shield is in said non-shielded position for biasing said shield to said shielded position.

20. A needle safety device as in claim 19, wherein said needle cannula lock in said first position exerts a biasing force against said needle cannula, biasing said shield toward the non-shielded position, and said biasing element exerts a biasing force greater than said biasing force exerted by said cannula lock for maintaining said shield biased toward the shielded position.

21. A safety assembly comprising:
a medical including a proximal end and a distal end;
a needle extending from the distal end of the medical device, said needle including a distal end having a puncture tip;
a laterally extending arm adjacent the distal end of said medical device and extending laterally from the medical device;
a laterally extending lever extending laterally from the distal end of the medical device;
a hinge section formed at the intersection of said laterally extending arm and said laterally extending lever and defining an acute angle therebetween, said hinge section further defining a pivot axis for pivotal movement of said laterally extending lever with respect to said laterally extending arm between a first position and a second position with said second position defining an angle which is more acute than said first position; and
a shield integral with said laterally extending lever and extending toward the distal end of said needle for encompassing said distal end of said needle when said hinge is in said first position,
wherein pivotal movement of said laterally extending lever from said first position to said second position causes biasing energy to accumulate between said laterally extending arm and said laterally extending lever, said biasing energy biasing said laterally extending lever to said first position for encompassing said distal end of said needle.

22. A safety assembly as in claim 21, wherein said biasing energy accumulates in a spring element causing a bending moment between said laterally extending arm and said laterally extending lever.

23. A safety assembly as in claim 22, wherein said spring element comprises a torsion spring.

24. A safety needle assembly as in claim 21, wherein said biasing energy accumulates in the hinge section.

25. A safety needle assembly as in claim 21, wherein said hinge section comprises a leaf spring.
